(19) United States
(12) Patent Application Publication
(10) Pub. No.: US 2022/0280952 A1
(43) Pub. Date: Sep. 8, 2022

(54) FLUID DISPOSING SYSTEM

(71) Applicant: CYTODX INC., Daejeon (KR)

(72) Inventors: Joseph SUNOO, Seongnam-si (KR); Min Pyo HONG, Suwon-si (KR); Sin Uk RYU, Seoul (KR)

(73) Assignee: CYTODX INC., Daejeon (KR)

(21) Appl. No.: 17/486,147

(22) Filed: Sep. 27, 2021

(30) Foreign Application Priority Data
Mar. 8, 2021 (KR) 10-2021-0030111

(51) Int. Cl. B04B 5/04; B04B 5/12; B04B 5/10; B03C 1/28; G01N 33/543; G01N 1/40; B01L 3/00; B01L 3/02; B01L 9/00; B01L 9/06; B01L 7/00; B03C 1/30

(52) U.S. Cl. CPC B04B 5/0442; B04B 5/12; B04B 5/10; B03C 1/288; G01N 33/54326; G01N 1/4077; B01L 3/52; B01L 3/021; B01L 9/54; B01L 9/06; B01L 9/50; B01L 7/00; B03C 1/30; B03C 2201/18; B03C 2201/26; B01L 2300/1822

(57) ABSTRACT

Disclosed is a fluid disposing system including a centrifugal separator that centrifugally separates a liquid that is supplied, a reagent supply module provided on one side of the centrifugal separator and that supplies a reagent to the centrifugal separator, a pipetting module provided at an upper portion of the centrifugal separator and that feeds a fluid to the centrifugal separator, a feeding module coupled to one side of the pipetting module, and that moves the pipetting module in an X axis direction and a Y axis direction corresponding to a horizontal direction, and a Z axis direction that is perpendicular to the horizontal direction, and a separation module including a magnetic bead for magnetically separating a material that has been primarily separated by the centrifugal separator.

### CROSS-REFERENCE TO RELATED APPLICATION(S)

This application is based on and claims priority under 35 U.S.C. § 119 to Korean Patent Application No. 10-2021-0030111, filed on Mar. 8, 2021, in the Korean Intellectual Property Office, the disclosure of which is incorporated by reference herein its entirety.

### BACKGROUND

### 1. Field

The disclosure relates to a fluid disposing system, and more particularly, to a fluid disposing system including a centrifugal separator.

### 2. Description of Related Art

A centrifugal separator may be used to extract peripheral blood mononuclear cells (PBMCs) or circulating tumor cells (CTCs) from blood. However, because an extremely small number of PBMCs or CTCs are present in blood and the cells may die if they are not separated within 24 hours after the blood of a person is collected, it is necessary to promptly and accurately extract the cells.

However, according to the conventional technology, because a reagent, a magnet, and a separator are used to separate CTCs and the like and a person directly intervenes with the separation process, a deviation of the result cannot be avoided according to the ability of the person that intervenes with the separation process, and there is a limit in the repetitiveness and the precision of the separation process.

For example, according to the conventional technology, they are extracted by injecting a floating density gradient material and blood into a container such as a conical tube for centrifugal separation, and inserting an extraction unit such as a pipette to a portion, at which the separated PBMCs are located. However, because the PBMCs or the CTCs may be easily lost as the blood and the density gradient material injected before the centrifugal separation and there is a limit in manually accurately inserting the extraction unit into the portion, at which the PBMCs are located, it is difficult to quantitatively extract the PBMCs or the CTCs.

Furthermore, according to the conventional technology, to extract target cells of high purity, a secondary centrifugal separation may be performed by extracting only a specific material after a primary centrifugal separation. However, there is a limit in accurately and promptly performing a process of carrying the primarily centrifugally separation material to another centrifugal separator or another chamber of centrifugal separation, for the secondary centrifugal separation.

### SUMMARY

Aspects of the disclosure are to address at least the above-mentioned problems and/or disadvantages and to provide at least the advantages described below. Accordingly, an aspect of the disclosure is to remarkably improve a repetitiveness and a precision of a separation process by implementing a complete automation in a separation process using a separator.

In accordance with an aspect of the disclosure, a fluid disposing system includes a centrifugal separator that centrifugally separates a liquid that is supplied, a reagent supply module provided on one side of the centrifugal separator and that supplies a reagent to the centrifugal separator, a pipetting module provided at an upper portion of the centrifugal separator and that feeds a fluid to the centrifugal separator, a feeding module coupled to one side of the pipetting module, and that moves the pipetting module in an X axis direction and a Y axis direction corresponding to a horizontal direction, and a Z axis direction that is perpendicular to the horizontal direction, and a separation module including a magnetic bead for magnetically separating a material that has been primarily separated by the centrifugal separator.

The centrifugal separator may include a rotor body rotated about a rotary shaft extending in an upward/downward direction, a first chamber coupled to one side of the rotor body and having a first space in an interior thereof, and a second chamber coupled to an opposite side of the rotor body and having a second space in an interior thereof, and a size of the first space and a size of the second space may be different.

The separation module may include a first tube rack provided on one side of the centrifugal separator and having a space, in which a tube is accommodated, and a second tube rack provided on one side of the centrifugal separator and having a space, in which a tube is accommodated, and the first tube rack and the second tube rack may face each other.

The fluid disposing system may further include a tip disposing unit provided on one side of the first tube rack and the second tube rack, the tip disposing unit may be separated from the centrifugal separator while the first tube rack and the second tube rack being interposed therebetween, and the tip disposing unit may include a tip accommodating member having an interior space for accommodating a tip, and a tip ejector provided at an upper portion of the tip accommodating member.

The fluid disposing system may further include a tip rack provided on one side of the tip disposing unit and having a space for accommodating the tip, and the tip rack may be spaced apart from the first tube rack and the second tube rack while the tip disposing unit being interposed therebetween.

The fluid disposing system may further include a shaker provided on one side of the second tube rack and provided adjacent to the second tube rack.

The fluid disposing system may further include a liquid disposing unit provided on one side of the tip disposing unit or one side of the shaker and provided adjacent to the tip disposing unit or the shaker.

The fluid disposing system may further include a sorter unit provided on one side of the liquid disposing unit, and the sorter unit may be spaced apart from the shaker while the liquid disposing unit being interposed therebetween.

The pipetting module may include a gripper that grips a tube, a large-capacity pipette part coupled to a lower portion of the gripper, and a small-capacity pipette part provided on one side of the large-capacity pipette part, and the large-capacity pipette part and the small-capacity pipette part may be integrally movable in an upward/downward direction.

The separation module may include a Peltier element provided at a lower end of the second tube rack, and the second tube rack may include a magnetic block, and a temperature measuring member.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a perspective view of a system of the disclosure;

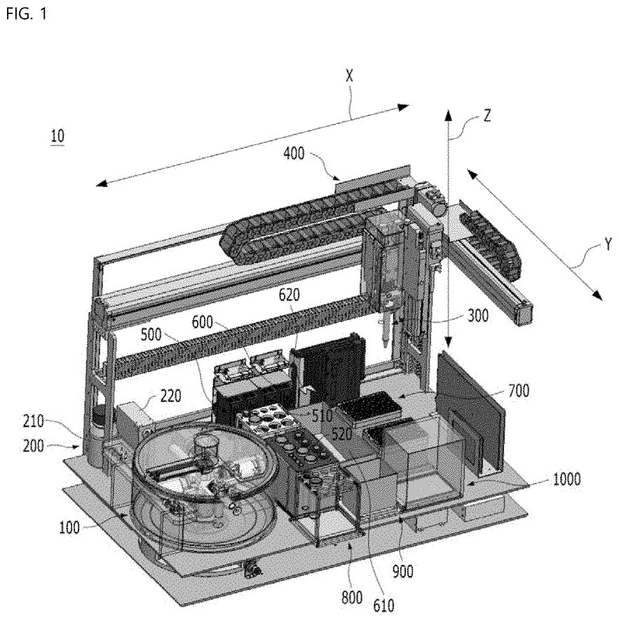

FIG. 2 is a perspective view of a centrifugal separator of the disclosure;

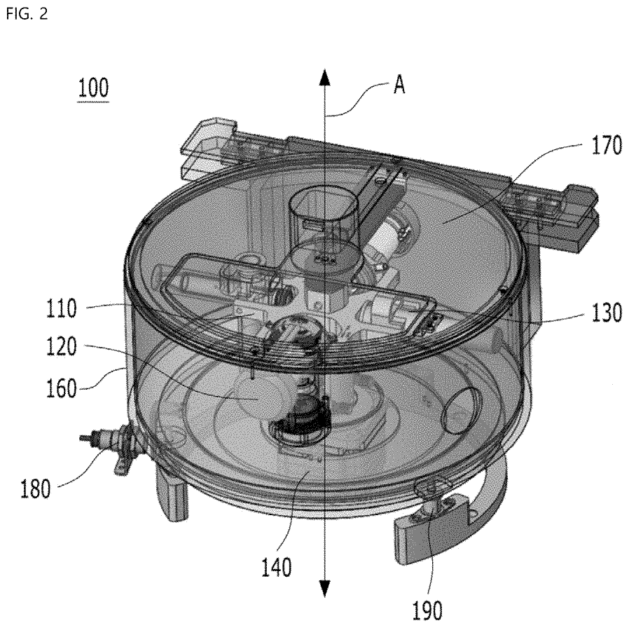

FIG. 3 is a perspective view of a pipetting module of the disclosure;

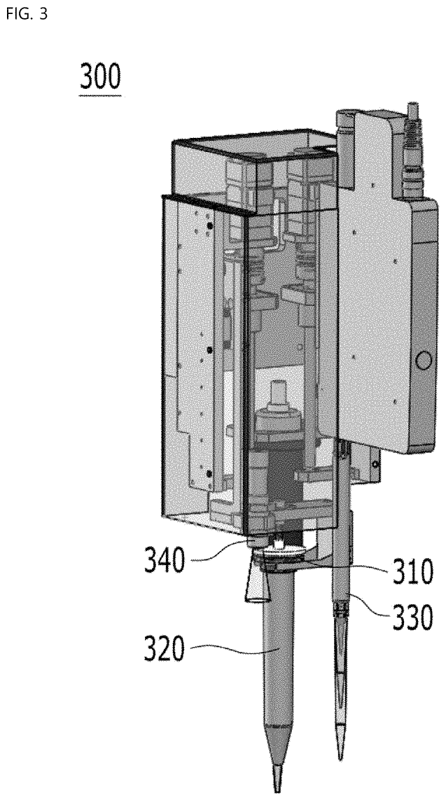

FIG. 4 is a perspective view of a second tube rack of the disclosure; and

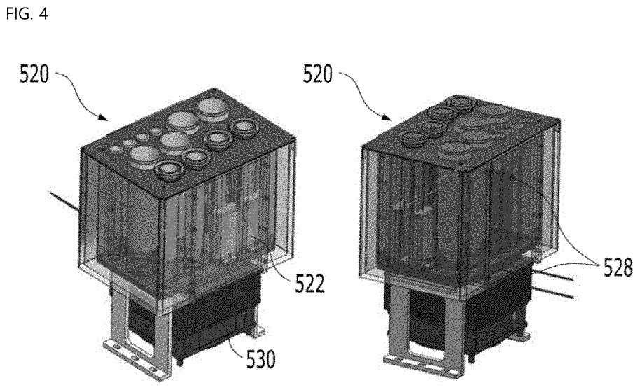

FIG. 5 is a perspective view of a magnetic block of the disclosure.

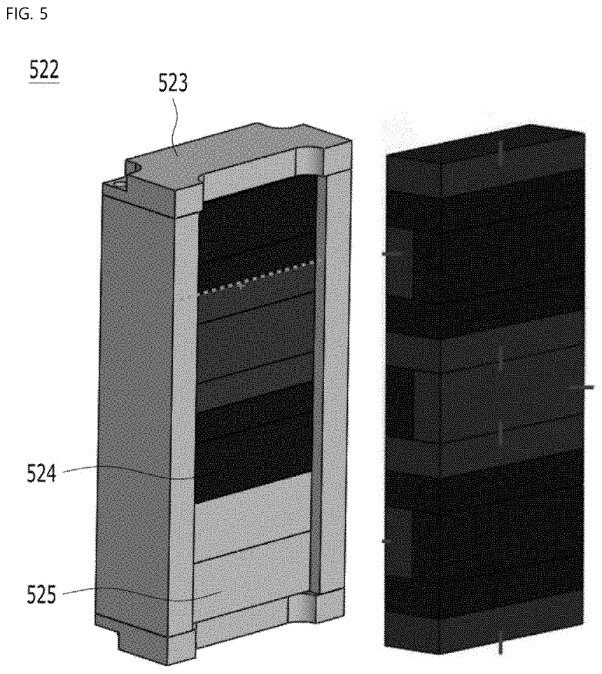

### DETAILED DESCRIPTION

Hereinafter, various embodiments of the disclosure will be described with reference to the accompanying drawings. It should be understood that the disclosure is not limited to specific embodiments and includes various modifications, equivalents, and/or alternatives of the embodiments of the disclosure. With regard to the description of drawings, similar components may be denoted by similar reference numerals.

In the disclosure disclosed herein, the expressions “have”, “may have”, “include” and “comprise”, or “may include” and “may comprise” used herein indicate existence of corresponding features (e.g., elements such as numeric values, functions, operations, or components) but do not exclude presence of additional features.

In the disclosure disclosed herein, the expressions “A or B”, “at least one of A or/and B”, or “one or more of A or/and B”, and the like used herein may include any and all combinations of one or more of the associated listed items. For example, the term “A or B”, “at least one of A and B”, or “at least one of A or B” may refer to all of the case (1) where at least one A is included, the case (2) where at least one B is included, or the case (3) where both of at least one A and at least one B are included.

According to the situation, the expression “configured to” used herein may be used as, for example, the expression “suitable for”, “having the capacity to”, “designed to”, “adapted to”, “made to”, or “capable of”. The term “configured to” must not mean only “specifically designed to” in hardware.

Terms used in this specification are used to describe specified embodiments of the disclosure and are not intended to limit the scope of the disclosure. The terms of a singular form may include plural forms unless otherwise specified. Unless otherwise defined herein, all the terms used herein, which include technical or scientific terms, may have the same meaning that is generally understood by a person skilled in the art. It will be further understood that terms, which are defined in a dictionary and commonly used, should also be interpreted as is customary in the relevant related art and not in an idealized or overly formal detect unless expressly so defined herein in various embodiments of the disclosure. In some cases, even if terms are terms which are defined in the specification, they may not be interpreted to exclude embodiments of the disclosure.

The embodiments disclosed herein are provided to describe the technical contents or for understanding of the technical contents, and the scope of the disclosure is not limited thereto. Accordingly, the scope of the disclosure should be construed to include all changes or various embodiments based on the technical spirit of the disclosure.

Hereinafter, preferred embodiments of the disclosure will be described in detail. Prior to the description of the disclosure, it will be noted that the terms and wordings used in the specification and the claims should not be construed as general and lexical meanings, but should be construed as the meanings and concepts that agree with the technical spirits of the disclosure, based on the principle stating that the concepts of the terms may be properly defined by the inventor(s) to describe the disclosure in the best manner.

Therefore, because the examples described in the specification and the configurations illustrated in the drawings are merely for the preferred embodiments of the disclosure but cannot represent all the technical sprints of the disclosure, it should be understood that various equivalents and modifications that may replace them can be present.

Throughout the specification, when it is described that a part includes an element, it may mean that the part may further include second element without excluding the second element unless a specially contradictory description is made.

Hereinafter, the disclosure will be described in detail.

As illustrated in the drawing, the fluid disposing system 10 according to the disclosure may include a centrifugal separator 100 configured to centrifugally separate a liquid that is supplied, a reagent supply module 200 provided on one side of the centrifugal separator 100 and configured to supply a reagent to the centrifugal separator 100, a pipetting module 300 provided at an upper portion of the centrifugal separator 100 and configured to feed a fluid to the centrifugal separator 100, a feeding module 400 coupled to one side of the pipetting module 300, and configured to move the pipetting module 300 in an X axis direction and a Y axis direction corresponding to a horizontal direction, and a Z axis direction that is perpendicular to the horizontal direction, and a separation module 500 including a magnetic bead for magnetically separating a material that has been primarily separated by the centrifugal separator 100. As an example, the above-described reagent may be a density gradient material. The drawing illustrates that the separator 100 is provided at a left end of the fluid disposing system 10 and the reagent supply module 200 is provided adjacent to the centrifugal separator 100 on one side of the separator 100. Because the pipetting module 300 may be moved in a horizontal direction and a vertical direction by the feeding module 400, as will be described below, the pipetting module 300 may inject and discharge a fluid while moving the centrifugal separator 100 and the like and feeding a tube, in a process according to the fluid disposing system 10 according to the disclosure.

The centrifugal separator 100 may be a configuration of separating a component that is to be extracted from blood and the like in a centrifugal separation scheme.

In more detail, the centrifugal separator 100 may include the rotor body 110 rotated about a rotary shaft extending in an upward/downward direction, the first chamber 120 coupled to one side of the rotor body 110 and having a first space in an interior thereof, and the second chamber 130 coupled to an opposite side of the rotor body 110 and having a second space in an interior thereof, and may further include a motor 140. The rotor body 110 may be coupled to the motor 140 to be rotated about the rotary shaft extending in the vertical direction. The first chamber 120 and the second chamber 130 may be provided in a circumferential area of the rotor body 110. The first chamber 120 and the second chamber 130 may perform different functions. For example, while a primary centrifugal separation process is performed in the first chamber 120, a secondary centrifugal separation process is performed in the second chamber 130. Accordingly, according to the disclosure, two centrifugal separation processes having different processes may be performed in one centrifugal separator 100.

Furthermore, a plurality of first chambers 120 and a plurality of second chambers 130 may be provided. In the drawings, as an example, two first chambers 120 and two second chambers 130 may be provided, and the first chambers 120 and the second chambers 130 may be alternately provided along a circumference of the rotor body 110, and it is illustrated that the first chambers 120 and the second chambers 130 are disposed at an equal interval.

Meanwhile, according to the disclosure, a size of the first space formed by the first chamber 120 and a size of the second space formed by the second chamber 130 may be different. For example, the size of the first space may be larger than the size of the second space. Furthermore, according to the disclosure, the centrifugal separator 100 may further include a separate weight balancing member (not illustrated) such that an overall center of weight of structures that are rotated while the centrifugal separator 100 is operated by driving the motor 140 is located on the rotary shaft.

The centrifugal separator 100 may further include a rotary union that passes through a central area of the rotor body 110. The rotary union may include an inner race and an outer race, and may have a structure, in which the inner race and the outer race are rotated relative to each other. As an example, in the rotary union, the outer race and the rotor body 110 may be rotated but the inner race may be fixed when the motor 140 is operated. To achieve this, the outer race may be coupled to the rotor body 110.

Meanwhile, the rotary union may be a configuration that also functions to deliver the liquid to the first chamber 120 and the second chamber 130. Then, in the rotary union, a path for delivering the liquid to the chambers may have a single path. In this case, an overall configuration of the centrifugal separator 100 may become simple without being restricted by a limit in a rotational speed of the centrifugal separator 100.

Furthermore, the centrifugal separator 100 may further include a case 160 that accommodates the rotor body 110 and an upper portion of which is opened, an automatic door 170 coupled to an upper area of the case 160 and that opens and closes an interior space of the case 160, a vibration detecting unit 180 that detects vibration of the centrifugal separator 100 in a centrifugal separation process according to driving of the motor 140, and a damper 190 for reducing vibration generated by the centrifugal separator 100.

Meanwhile, the reagent supply module 200 may be provided adjacent to the central separator 100 such that a length of a path, in which the reagent (for example, a density gradient material) supplied to the centrifugal separator 100 is fed, may be minimized. The drawing illustrates that the centrifugal separator 100 and the reagent supply module 200 are made to face each other by providing the reagent supply module 200 on one side of the centrifugal separator 100 in the Y axis direction. Furthermore, the reagent supply module 200 may include a reagent keeping member 210 for keeping the reagent, and a pump member 220 for supplying the reagent accommodated in the reagent keeping member 210 to an outside.

Meanwhile, the separation module 500 may further include the first tube rack 510 that is provided on one side of the centrifugal separator 100 and has a space, in which a tube is accommodated, and the second tube rack 520 that is provided on one side of the centrifugal separator 100 and has a space, in which a tube is accommodated. Then, the first tube rack 510 and the second tube rack 520 may face each other. The drawings illustrate as an example that the first tube rack 510 and the second tube rack 520 are provided on a right side of the centrifugal separator 100 to face the centrifugal separator 100 and the first tube rack 510 and the second tube rack 520 are provided in the Y axis direction.

For example, tubes for buffering for dilution of blood, extracting a specific material, such as a PBMC or plasma, and keeping the material in a concentrated state may be disposed in the first tube rack 510. Furthermore, although not illustrated in the drawings, the first tube rack 510 may further include an indexing apparatus for identifying a sample.

As will be described below, it may be preferable that a moving line, along which the sample is injected into the chamber or the tube provided in the centrifugal separator 100 and is fed by using the pipetting module 300 provided in the fluid disposing system 10 according to the disclosure, is minimized. Accordingly, as described above, it is more preferable that the first tube rack 510 and the second tube rack 520 are provided on one side of the centrifugal separator 100 to face the centrifugal separator 100.

Referring to the drawings continuously, the separation module 500 of the fluid disposing system 10 according to the disclosure may further include a Peltier element provided at a lower portion of the second tube rack 520, and the second tube rack 520 may further include a magnetic block 522 and a temperature measuring member 528.

In comparison with the first tube rack 510, the second tube rack 520 may be maintained in a low temperature state. For example, the second tube rack 520 may be maintained at a temperature of 3 degrees to 8 degrees Celsius.

To achieve this, a Peltier element 530 may be provided at a lower portion of the second tube rack 520. The Peltier element 530 may be a configuration capable of cooling one side by implementing a temperature difference between opposite ends by using a thermoelectric effect or a Peltier effect. That is, when an electric current flows in the Peltier element 530, a low temperature area of the Peltier element 530 may be configured to contact the second tube rack 520. Accordingly, the second tube rack 520 may be maintained in a low temperature state. For example, an outer case of the second tube rack 520 may be formed of a metallic material such as aluminum. In this case, heat of the second tube rack 520 may promptly flow to the Peltier element 530, and thus, the second tube rack 520 also may be promptly cooled. Meanwhile, an insulation member for interrupting heat transfer to an outside may be further included around an outer case of the second tube rack 520.

The magnetic block 522 may be a configuration for separating a magnetic bead coated with a specific antibody from a material that is to be extracted, in a process of extracting a material, such as a CTC. To achieve this, the magnetic block 522 may include an outer member 523, and a magnet member 524 provided in an interior of the outer member 523. The magnet member 524 provided in the magnetic block 522 may include an N pole area and an S pole area, and the N pole area and the S pole area may have a specific disposition structure that may have an optimum magnetic flux for separating the magnetic bead. Furthermore, the magnetic block 522 may further include a metal member 525 provided in a lower area of the magnet member 524 to correspond to a height of the tube, from which the magnetic bead is separated.

Referring to the drawings continuously, the fluid disposing system 10 according to the disclosure may further include the tip disposing unit 600 provided on one side of the first tube rack 510 and the second tube rack 520.

The tip disposing unit 600 may be a configuration of processing the tip in a process of feeding, injecting, and discharging a material, such as a sample, by using the fluid disposing system 10 according to the disclosure.

Then, according to the disclosure, the tip disposing unit 600 may be spaced apart from the centrifugal separator 100 while the first tube 510 and the second tube rack 520 being interposed therebetween. The drawings illustrate that the tip disposing unit 600 is provided on a right side of the first tube rack 510 and the second tube rack 520 and is separated from the centrifugal separator 100 provided on a left side of the first tube rack 510 and the second tube rack 520.

Meanwhile, the tip disposing unit 600 may include a tip accommodating member 610 having an interior space for accommodating the tip, and a tip ejector 620 provided at an upper portion of the tip accommodating member 610. Accordingly, according to the disclosure, the used tip may drop into the interior space of the tip accommodating member 610 after being separated from the pipetting module 300 by the tip ejector 620.

Referring to the drawings continuously, the fluid disposing system 10 according to the disclosure may further include the tip rack 700 that is provided on one side of the tip disposing unit 600 and has a space, in which the tip may be accommodated.

The tip rack 700 is a configuration provided with the tip such that a new tip may be mounted on the pipetting module 300, and as an example, the tip rack 700 may be spaced apart from the first tube rack 510 and the second tube rack 520 while the tip disposing unit 600 being interposed therebetween. The drawings illustrate that the tip rack 700 is provided on a right side of the tip disposing unit 600 to be spaced apart from the first tube rack 510 and the second tube rack 520 provided on a left side of the tip disposing unit 600. In this case, because the used tip does not pass above the tip rack 700 provided with a tip that has not been used yet in a process of feeding the used tip from the centrifugal separator 100, the first tube rack, and the second tube rack to the tip disposing unit 600, the tip provided in the tip rack 700 may be prevented from being contaminated in advance.

However, unlike the illustration of the drawings, locations of the tip rack 700 and the tip disposing unit 600 may be exchanged. That is, unlike the illustration of the drawings, the tip disposing unit 600 may be provided on a right side of the tip rack 700 to be spaced apart from the first tube rack 510 and the second tube rack 520 provided on a left side of the tip rack 700.

Meanwhile, the fluid disposing system 10 according to the disclosure may further include a shaker 800 that is provided on one side of the second tube rack 520 and is provided adjacent to the second tube rack 520. The drawings illustrate that the shaker 800 is provided on one side of the second tube rack 520 in the Y axis direction to face the second tube rack 520. The shaker 800 may be a configuration for forming flows in a solution in the tube such that a material (for example, cells) in the solution accommodated in the tube of the second tube rack 520.

Referring to the drawings continuously, the fluid disposing system 10 according to the disclosure may further include a liquid disposing unit 900 that is provided on one side of the tip disposing unit 600 or one side of the shaker 800 and is provided adjacent to the tip disposing unit 600 or the shaker 800. The drawing illustrates that the liquid disposing unit 900 is provided on a right side of the shaker 800 and thus the liquid disposing unit 900 faces the shaker 800. Furthermore, the drawing illustrates the liquid disposing unit 900 is provided on a lower side of the tip disposing unit 600 in the Y axis direction and thus is provided adjacent to the tip disposing unit 600. The liquid disposing unit 900 may be a configuration for keeping a solution that is discarded after being used in a process of operating the fluid disposing system 10.

Referring to the drawings continuously, the fluid disposing system 10 according to the disclosure may further include a sorter unit 100 provided on one side of the liquid disposing unit 900. Then, the sorter unit 1000 may be spaced apart from the shaker 800 while the liquid disposing unit 900 being interposed therebetween. The drawings illustrate that the sorter unit 1000 is provided on a right side of the liquid disposing unit 900 and is spaced apart from the shaker 800 provided on a left side of the liquid disposing unit 900. The sorter unit 1000, for example, may be a configuration of separating specific cells, such as CTCs, from blood and the like. In particular, the sorter unit 1000 may be a fluorescence activated cell sorter (FACS) for improving a purity of cells, such as CTCs, which are separated from the blood. The sorter 1000 may be spaced apart from the centrifugal separator 100, the first tube rack, and the second tube rack by a specific distance.

Meanwhile, referring to the drawings continuously, the pipetting module 300 for feeding a fluid may include the gripper 310 configured to grip the tube, the large-capacity pipette part 320 coupled to the lower portion of the gripper 310, and the small-capacity pipette part 330 provided on one side of the large-capacity pipette part 320.

It is necessary to feed fluids or materials having various kinds of volumes in a fluid disposing process including the centrifugal separation process, and according to the disclosure, the large-capacity pipette part 320 and the small-capacity pipette part 330 are provided so that the fluids or the materials having various kinds of volumes may be smoothly fed.

In particular, according to the disclosure, the large-capacity pipette part 320 and the small-capacity pipette part 330 may be coupled to one shaft that may be moved in an upward/downward direction. That is, the large-capacity pipette part 320 and the small-capacity pipette part 330 may be integrally movable in the upward/downward direction. Accordingly, according to the disclosure, the large-capacity pipette part 320 and the small-capacity pipette part 330 may be simultaneously manipulated by moving the one shaft in the upward/downward direction.

Meanwhile, the gripper 310 may be a configuration for directly gripping the tube, and as illustrated in the drawings, may be provided adjacent the large-capacity pipette part 320. Meanwhile, the pipetting module 300 may further include an ultrasonic wave sensor 340 for detecting whether the tube is gripped by the gripper 310. The drawings illustrate that a distance sensor 340 is provided at an upper portion of the gripper 310.

A repetitiveness and a precision of a separation process may be remarkably improved by implementing a complete automation in a separation process using a separator.

The above description is a simple exemplification of the technical spirit of the disclosure, and the disclosure may be variously corrected and modified by those skilled in the art to which the disclosure pertains without departing from the essential features of the disclosure. Therefore, the disclosed embodiments of the disclosure do not limit the technical spirit of the disclosure but are illustrative, and the scope of the technical spirit of the disclosure is not limited by the embodiments of the disclosure. The scope of the disclosure should be construed by the claims, and it will be understood that all the technical spirits within the equivalent range are fall within the scope of the disclosure.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. A fluid disposing system comprising:
a centrifugal separator configured to centrifugally separate a liquid that is supplied;
a reagent supply module provided on one side of the centrifugal separator and configured to supply a reagent to the centrifugal separator;
a pipetting module provided at an upper portion of the centrifugal separator and configured to feed a fluid to the centrifugal separator;
a feeding module coupled to one side of the pipetting module, and configured to move the pipetting module in an X axis direction and a Y axis direction corresponding to a horizontal direction, and a Z axis direction that is perpendicular to the horizontal direction; and
a separation module including a magnetic bead for magnetically separating a material that has been primarily separated by the centrifugal separator.

2. The fluid disposing system of claim 1, wherein the centrifugal separator includes:
a rotor body rotated about a rotary shaft extending in an upward/downward direction;
a first chamber coupled to one side of the rotor body and having a first space in an interior thereof; and
a second chamber coupled to an opposite side of the rotor body and having a second space in an interior thereof, and
a size of the first space and a size of the second space are different.

3. The fluid disposing system of claim 1, wherein the separation module includes:
a first tube rack provided on one side of the centrifugal separator and having a space, in which a tube is accommodated; and
a second tube rack provided on one side of the centrifugal separator and having a space, in which a tube is accommodated, and
wherein the first tube rack and the second tube rack face each other.

4. The fluid disposing system of claim 3, further comprising:
a tip disposing unit provided on one side of the first tube rack and the second tube rack,
wherein the tip disposing unit is separated from the centrifugal separator while the first tube rack and the second tube rack being interposed therebetween, and wherein the tip disposing unit includes:
a tip accommodating member having an interior space for accommodating a tip; and
a tip ejector provided at an upper portion of the tip accommodating member.

5. The fluid disposing system of claim 4, further comprising:
a tip rack provided on one side of the tip disposing unit and having a space for accommodating the tip,
wherein the tip rack is spaced apart from the first tube rack and the second tube rack while the tip disposing unit being interposed therebetween.

6. The fluid disposing system of claim 4, further comprising:
a shaker provided on one side of the second tube rack and provided adjacent to the second tube rack.

7. The fluid disposing system of claim 6, further comprising:
a liquid disposing unit provided on one side of the tip disposing unit or one side of the shaker and provided adjacent to the tip disposing unit or the shaker.

8. The fluid disposing system of claim 7, further comprising:
a sorter unit provided on one side of the liquid disposing unit, and
wherein the sorter unit is spaced apart from the shaker while the liquid disposing unit being interposed therebetween.

9. The fluid disposing system of claim 1, wherein the pipetting module includes:
a gripper configured to grip a tube;
a large-capacity pipette part coupled to a lower portion of the gripper; and
a small-capacity pipette part provided on one side of the large-capacity pipette part, and
wherein the large-capacity pipette part and the small-capacity pipette part are integrally movable in an upward/downward direction.

10. The fluid disposing system of claim 3, wherein the separation module includes:
a Peltier element provided at a lower end of the second tube rack, and
wherein the second tube rack includes:
a magnetic block; and
a temperature measuring member.
